(19) United States
(12) Patent Application Publication
(10) Pub. No.: US 2019/0159495 A1
(43) Pub. Date: May 30, 2019

(54) METHOD FOR SHORT-TIME KOJI PRODUCTION USING PRE-CULTURED FILAMENTOUS FUNGI

(71) Applicant: Yamasa Corporation, Choshi-shi (JP)

(72) Inventors: Yoshiyuki TOYOSHIMA, Choshi-shi (JP); Keitaro KUDO, Choshi-shi (JP); Yasunori MATSUURA, Choshi-shi (JP)

(21) Appl. No.: 16/203,246

(22) Filed: Nov. 28, 2018

(30) Foreign Application Priority Data
Nov. 29, 2017 (JP) 2017-228905

(51) Int. Cl. A23L 27/24; A23L 27/50; A23L 27/10; C12N 1/14

(52) U.S. Cl. CPC A23L 27/24; C12N 1/14; A23L 27/10; A23L 27/50

(57) ABSTRACT

An object of the present invention is to obtain Koji having an enzyme titer equal to or higher than that based on conventional methods without contamination of putrefactive bacteria within 30 hours which is a very short Koji production time.

The present invention provides a method for producing Koji for brewed foods, including the steps of: (1) obtaining pre-cultured seed Koji by pre-culturing Koji mold using a culture material with 500 g/L or less of bulk specific gravity, and (2) performing Koji production for 18 to 30 hours after inoculating the pre-cultured seed Koji into a protein raw material and/or a carbohydrate raw material.

### CROSS REFERENCE TO RELATED APPLICATIONS

This application claims the priority of Japanese Patent Application No. 2017-228905 filed on Nov. 29, 2017, which is incorporated by reference herein in its entirety.

### TECHNICAL FIELD

The present invention relates to a method for producing Koji with remarkably shortened Koji production time by pre-culturing seed Koji and obtaining germinated hyphae, and producing Koji which has a high enzyme titer and a low risk of contamination by various bacteria.

### BACKGROUND ART

In production of brewed foods such as soy sauce, miso and sake, it is very important to produce Koji by inoculating Koji mold (seed Koji) into a protein raw material and/or a carbohydrate raw material subjected to pretreatment such as steaming and roasting; germinating the Koji mold on the raw material; and then culturing the Koji mold thoroughly (Koji production process). For example, when producing soy sauce, steamed soybeans and roasted wheat are mixed as a raw material, and then “seed Koji” as a source of Koji mold is added to the raw material. The seed Koji means sporulated Koji mold or a culture material containing the sporulated Koji mold. The seed Koji can be produced by inoculating the Koji mold into pearl barley flakes or crushed wheat, barley or rice, or the like, and then sufficiently sporulating the inoculated Koji mold by culturing for about 72 to 120 hours. After mixing the seed Koji with grain as a raw material, the mixture is cultured for 3 to 4 days under the environment of high temperature and high humidity, resulting that “Koji” is obtained by growing the Koji mold thoroughly on the grain as the raw material.

If the Koji mold does not grow sufficiently during the Koji production, there is a risk of insufficient decomposition of protein or carbohydrate in the raw material. On the other hand, if the growth of the Koji mold is excessive, the temperature of the Koji rises too much due to the heat generated by the Koji mold, resulting of affecting the quality of the Koji or food. Therefore, in order to keep the growth of the Koji mold in a suitable state in the Koji production process, a large amount of energy is required to control, for example, the temperature of the culture environment within the appropriate range of the temperature of the Koji. Therefore, in order to solve the economic problem and to reduce the energy used, various methods for shortening the Koji production time have been studied.

For example, Patent Document 1 discloses, in the production of soy sauce Koji, a technique in which the Koji production time is shortened from 4 days to 3 days by a method for Koji production including steps of adding purely cultured seed Koji to sterilized kernels or crushed products thereof soaked with water and culturing it, then adding the cultured product to a raw material processed by general methods when hyphae of Koji mold is sufficiently propagated. However, this method takes at least three days to produce the Koji. Thus, it is difficult to shorten the Koji production time from three days to two days.

In China, 24-hour Koji production in brewing of soy sauce and miso has been widely used from the 1970's (Non-Patent Document 1). However, in such a short-time Koji production, there are quality problems such as immature incense in comparison with products by traditional brewing methods and Koji with low enzyme titer.

In addition, in order to shorten the Koji production time, methods for shortening the time from the addition of the seed Koji to the germination of spores of the Koji mold (about 4-8 hours) have been studied. For example, there are a method for placing spores of filamentous fungi in the presence of ethylene gas for 1 hour or longer (Patent Document 2), and a method for culturing seed Koji under 20 to 65% of moisture content (Patent Document 3). However, although the germination rate of the Koji mold improves in both cases, it has not been able to significantly shorten the Koji production time such as to shorten the Koji production time from three days to two days.

Furthermore, culturing with low microbial contamination even in an open system and shortening of Koji production time have been realized by increasing the use rate of seed Koji beyond about 1 part of seed Koji per 1,000 parts of raw material, which is often used in ordinary Koji production. Even in the traditional short-time Koji production in China, the seed Koji is used about two times as much as ordinary Koji production (Non-Patent Document 1). However, since cost of the seed Koji production is expensive, a production method for reducing the amount of the seed Koji used as much as possible and obtaining the maximum effect has been demanded.

In addition, since red Koji mold (Monascus genus), which is a filamentous fungus used for red liquor and pigment production, has weak growth, a preculture method using 0.05% by dry weight or more of the red mold used as seed Koji after increasing the number of the red mold in preculture in liquid medium has been used (Non-Patent Document 2). However, this method is related to the preculture in the liquid medium. Preculture in solid medium has not been done at all in the past.

### PRIOR ART DOCUMENT

### Patent Document

Patent Document 1: JP S45-30198 A
Patent Document 2: JP S48-48680 A
Patent Document 3: JP 2005-210903 A

### Non-Patent Document

Non-Patent Document 1: Journal of Brewing Society of Japan, Vol. 91, No. 7, pp. 478-482
Non-Patent Document 2: Kojigaku, Murakami (ed.), Brewing Society of Japan, 1986, pp. 474-478

### SUMMARY OF THE INVENTION

### Problems to be Resolved by the Invention

An object of the present invention is to obtain Koji having an enzyme titer equal to or higher than that based on conventional methods without contamination of putrefactive bacteria within 30 hours which is a very short Koji production time, and to produce a food having the same ingredients and organoleptic properties as those of conventional brewed foods using Koji.

### Means of Solving the Problems

As a result of extensive studies to solve the above-described problems, the inventors of the present invention have completed the present invention based on the discovery that it is possible not only to dramatically shorten Koji production time within 30 hours but also to obtain Koji having a high enzyme titer and to produce brewed foods equal to or higher than the conventional quality by using sufficiently germinated Koji mold as seed Koji, pre-cultured on a raw material having a low bulk specific gravity such as wheat bran.

### Effect of the Invention

As clearly shown in Examples below, the short-time Koji production method of the present invention is capable of shortening the culturing time greatly and obtaining Koji having a high enzyme titer. Furthermore, brewed foods obtained by using the Koji have the equal ingredients and organoleptic properties as those obtained by the conventional Koji production methods.

### BRIEF DESCRIPTION OF THE DRAWINGS

FIG. 1 shows the appearances of Koji when Koji production was performed by using pre-cultured seed Koji cultured in different preculture media.

FIG. 2 shows the relationships between Koji production time and Koji temperature when Koji were made using pre-cultured seed Koji cultured in preculture media with different bulk specific gravity.

### DETAILED DESCRIPTION OF THE INVENTION

### Definition

For convenience, certain terms employed in the context of the present disclosure are collected here. Unless defined otherwise, all technical and scientific terms used herein have the same meaning as commonly understood by one of the ordinary skill in the art to which this invention belongs. The singular forms “a”, “and”, and “the” are used herein to include plural referents unless the context clearly dictates otherwise.

Notwithstanding that the numerical ranges and parameters setting forth the broad scope of the invention are approximations, the numerical values set forth in the specific examples are described as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art.

### Method According to the Present Embodiment

The present invention relates to a method for producing Koji for brewed foods, including the steps of: (1) obtaining pre-cultured seed Koji by pre-culturing Koji mold using a culture material with 500 g/L or less of bulk specific gravity, and (2) performing Koji production for 18 to 30 hours after inoculating the pre-cultured seed Koji into a protein raw material and/or a carbohydrate raw material.

Each step is described in detail below. It should be noted that when there is no mention of “specific gravity”, it means bulk specific gravity “before water absorption”. The bulk specific gravity refers to specific gravity (g/L) at a level container of the raw material without being compressed in 1 L container.

### (1) Step of Obtaining Pre-Cultured Seed Koji by Pre-Culturing Koji Mold Using a Culture Material With 500 g/L or Less of Bulk Specific Gravity

In the first step, Koji mold is inoculated into preculture medium and then precultured to sufficiently germinate the Koji mold.

In the inoculation, a culture material usable for brewed foods is sufficiently sterilized by autoclaving or the like, water is absorbed into the raw material as necessary, and then desired Koji mold is inoculated into the culture material so as to be 1×10⁶ to 5×10⁶ per 1 g of the culture material in terms of the number of spores. In the case of using the culture material with the water, the water may be added to the culture material at a ratio of 0.2 L to 1.0 L, preferably 0.4 L to 0.7 L per 1 kg of the culture material. After the inoculation, the Koji mold is cultured at 20 to 35° C. for 18 to 70 hours, preferably 24 to 48 hours in order to sufficiently germinate the Koji mold. It can be determined whether the Koji mold has been sufficiently germinated, for example, by measuring the number of spores. When the number of spores measured by a hemocytometer is 1×10⁷/g or more, it can be judged that the Koji mold has been germinated sufficiently.

Fungi commonly used in the production of brewed foods can be appropriately used as the Koji mold to be inoculated. Specifically, genus Aspergillus such as Aspergillus oryzae, Aspergillus sojae, Aspergillus kawachii and Aspergillus awamori, genus Monascus such as Monascus purpureus, and genus Rhizopus can be used as the Koji mold. Among them, the Koji mold of the genus Aspergillus is preferable, and Aspergillus oryzae or Aspergillus sojae is more preferable.

In the culture of pre-cultured seed Koji in the present invention, as the culture material, a culture material with 500 g/L or less of bulk specific gravity that can be used for producing the Koji for brewed foods is used.

Specific examples of such a culture material include epidermal portion (germin portion) derived from one or more raw materials selected from, for example, beans, wheat and barley, gramineous millet, pseudo grains and potatoes. In detail, “beans” such as soybeans, common beans, green peas, broad beans and red beans, “wheat and barley” such as wheat, barley, rye and oat, “gramineous millet” such as corn, foxtail millet and common millet, “pseudo grains” such as buckwheat and quinoa, and “potatoes” such as sweet potatoes can be used, among which the epidermal portion of the wheat or the barley is particularly preferred, and the epidermis of the wheat or barley is more preferred. If the bulk specific gravity is within the above-mentioned numerical range, it may contain a portion other than the epidermis.

(2) Step of Performing Koji Production for 18 to 30 Hours After Inoculating the Pre-Cultured Seed Koji Into a Protein Raw Material and/or a Carbohydrate Raw Material.

Next, Koji production is performed using the resulting pre-cultured seed Koji. Specifically, the pre-cultured seed Koji obtained in the above step (1) is inoculated into the raw material including the protein material and/or the carbohydrate material. The amount of the pre-cultured seed Koji to be inoculated can be appropriately determined, and is preferably 20 to 100 parts of the pre-cultured seed Koji per 1000 parts of the raw material, and more preferably 30 to 80 parts of the pre-cultured seed Koji per 1000 parts of the raw material. That is, it is preferable that the pre-cultured seed Koji containing the Koji molds corresponding to 2×10⁸ spores or more, preferably 3×10⁸ spores or more in terms of the number of spores measured by a hemocytometer is mixed with 1000 g of the raw material.

The protein raw material may include beans such as soybeans, common peas and broad beans and/or various fishery products as raw materials for fish sauce. The carbohydrate raw material may include wheat, barley, other wheat and barley rice, and/or corn. Well-known raw material can be used according to a brewed food type.

For example, in the performing soy sauce Koji production according to a conventional method, Koji production is performed after mixing 20 to 100 parts of pre-cultured seed Koji with 1000 parts of raw materials based on steamed soybeans as a protein raw material and roasted, cracked wheat as a carbohydrate raw material.

Specifically, the Koji production is performed after mixing the protein raw material and/or the carbohydrate raw material with the pre-cultured seed Koji, and then culturing the mixture for 18 to 30 hours, preferably 22 to 28 hours under a condition where the temperature of Koji is about 24 to 30° C. In the Koji production, “teire”, which means that the Koji is stirred in order to suppress the rise of the temperature of the Koji, may be performed once or twice, if necessary, or may not be performed. Well-known Koji production apparatus such as a stationary culture type, a ventilation culture type or the like using a tray (“Koji-buta”), a rotating disk, a conveyer or the like or combination thereof if necessary can be used.

The Koji obtained as such can be used for producing desired brewed foods by a conventional method. For example, when a brewed food is soy sauce, soy sauce can be obtained by adding an appropriate amount of salt water with a salt concentration of 20 to 27% (w/v) to the Koji and then fermenting and aging them at 15 to 30° C. for 3 to 6 months.

The Koji production method of the present invention can be applied to the production of brewed foods based on Koji such as miso and alcoholic beverages other than soy sauce. In spite of greatly shortening the Koji production time by replacing the conventional Koji production process with the method of the present invention, brewed foods having characteristics equivalent to those of the conventional brewed foods can be obtained even in the case of producing any brewed food.

### EXAMPLES

Hereinafter, the present invention will be specifically described with reference to examples, but the present invention is not limited to these examples in any way.

### Example I

### Various Studies on Preculture

The culture medium and incubation time in pre-culture of Koji mold were determined.

### (1-1) Study of Preculture Medium—1

Preculture media A and B shown below were obtained as preculture medium.
[Preculture Medium A] Water (0.7 L) was added to 1 kg of wheat bran (seed coat portion of wheat) as a raw material to absorb water. The water-soaked wheat bran was steamed to prepare Medium A (bulk specific gravity of wheat bran before water absorption is 380 g/L).
[Preculture Medium B] Water (20 L) was added to 15 kg of defatted soybean to absorb water, and the mixture was steamed in a conventional manner. In addition, 15 kg of raw wheat was roasted and cracked according to a conventional method. The steamed soybeans were mixed with the cracked wheat at equal weight to prepare Medium B (bulk specific gravity of defatted soybeans and wheat before water absorption is 670 g/L).

### Example 1

Aspergillus oryzae spores at 3×10⁶ spores/g were inoculated into the Medium A and cultured at 28° C. for 24 hours (preculture). The obtained pre-cultured seed Koji was mixed with the Medium B at a ratio of 5% per the weight of the Medium B, and then Koji-production was performed for 24 hours.

### Comparative Example 1

Aspergillus oryzae spores at 3×10⁶ spores/g were inoculated into the Medium B and cultured at 28° C. for 24 hours (preculture). The obtained pre-culture material was mixed with a fresh Medium B at a ratio of 5% per the weight of the Medium B, and Koji-production was performed for 24 hours.

### Control 1

Without carrying out the preculture of Comparative Example 1, Aspergillus oryzae spores at 3×10⁶ spores/g were directly applied to the Medium B, and then Koji-production was performed for 24 hours.

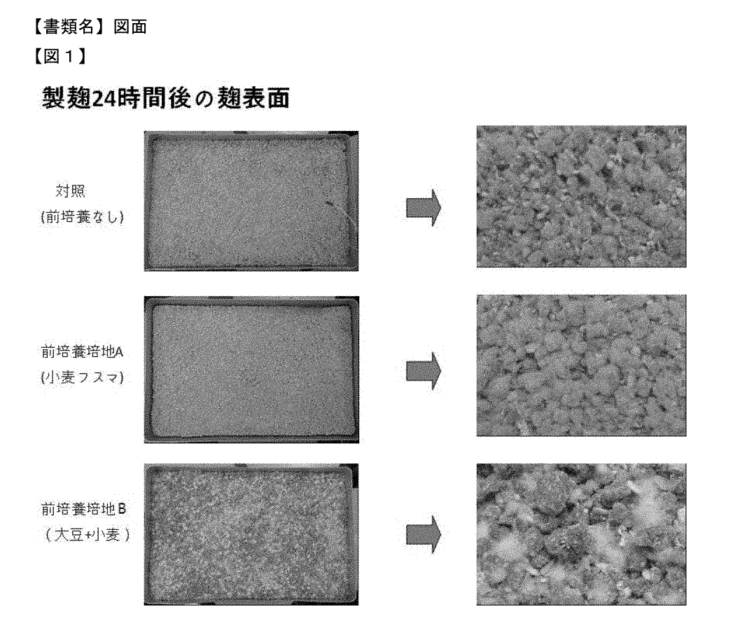

The results are shown in FIG. 1. In Comparative Example 1, the growth of hyphae of Aspergillus oryzae was insufficient, so that the Koji mold did not uniformly spread throughout the medium, which was inappropriate as Koji. On the other hand, in Example 1 in which the wheat bran was used as the preculture medium, the hyphae grew well to the same extent as Control 1, and favorable Koji could be obtained.

### (1-2) Study of Preculture Medium—2

Roasted/cracked wheat was mixed with wheat bran at a ratio as shown in the following Table 1 to obtain Preculture Media A to D (Media A and B are the same as the media described in the above (1-1)).

TABLE 1

| | | | Medium D | Medium B |
|---|---|---|---|---|
| | Medium A | Medium C | (Comparative | (Comparative |
| | (Example 2) | (Example 3) | Example 2) | Example 3) |
| Wheat bran (%) | 100 | 50 | 25 | Soybean 50% |
| Cracked wheat | 0 | 50 | 75 | Wheat 50% |
| (%) | | | | |
| Bulk specific | 380 | 500 | 600 | 670 |
| gravity (g/L) | | | | |

After performing the preculture and the Koji production in the same manner as Example 1 using Preculture Media A (Example 2), Media B (Comparative Example 3), Media C (Example 3), Media D (Comparative Example 2), the growth conditions of the Koji mold were compared using, as an index, the time until the temperature of the Koji was reached to 35° C. Since the Koji mold emits heat during the growth, the higher the temperature increases, the more the Koji grows actively, which indicates that the Koji is favorable. In particular, it is preferable that the temperature rises rapidly at the start of the Koji production.

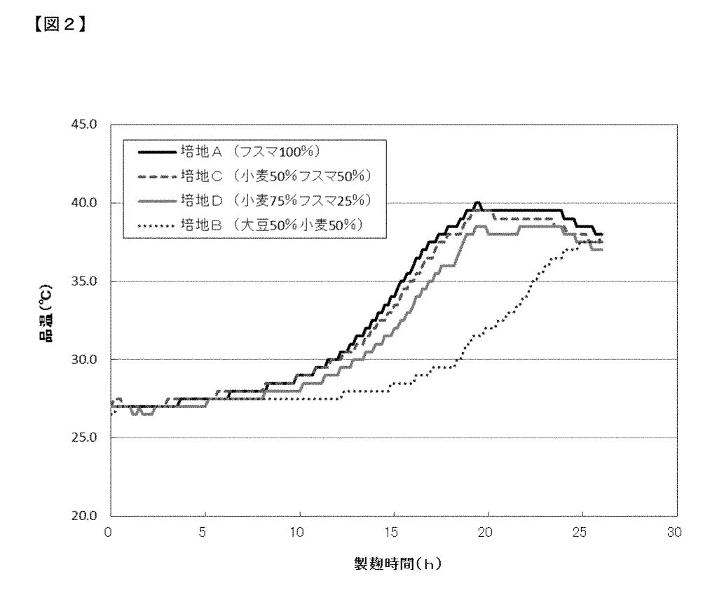

As a result shown in FIG. 2, the time to be reached to 35° C. was about 15 hours after the start of the culture in Example 2 (bulk specific gravity of raw material is 380 g/L) and Example 3 (bulk specific gravity is 500 g/L). On the other hand, the time to be reached to 35° C. was 17 hours in Comparative Example 2 (bulk specific gravity is 600 g/L) and 22 hours in Comparative Example 3 (bulk specific gravity is 670 g/L), which are not suitable for the Koji production because the increasing of the temperature indicating the growth of Aspergillus oryzae was greatly delayed.

Therefore, it was found that it was suitable to use, as a preculture medium, a culture material with 500 g/L or less of bulk specific gravity which is a low bulk specific gravity.

### (1-3) Examination of Preculture Time

The pre-cultured seed Koji were produced at 28° C. under the preculture times of Example 1 set to 18, 20, 22 and 24 hours by using the steamed, water-soaked wheat bran which was prepared by adding 0.7 L of water to 1 kg of the wheat bran and steaming them, as a preculture medium (Medium A). As Comparative Example 4, the preculture material was produced at 28° C. under preculture time of Example 1 set to 16 hours. The number of spores (the number of spores/g) in the pre-cultured seed Koji and preculture material at the end of the preculture were measured with a hemocytometer. Furthermore, the time until Koji temperature was reached to 35° C. was measured during the Koji production process performed in the same manner as the above (1-1) using each pre-cultured seed Koji and preculture material.

The result is shown in Table 2 below.

TABLE 2

| | Example | Example | Example | Example | Comparative |
|---|---|---|---|---|---|
| | 4 | 5 | 6 | 7 | Example 4 |
| Preculture | 24 hours | 22 hours | 20 hours | 18 hours | 16 hours |
| time | | | | | |
| The number | 8 × 10⁷ | 5 × 10⁷ | 3 × 10⁷ | 1 × 10⁷ | 2 × 10⁶ |
| of spores | | | | | |
| (the number | | | | | |
| of spores/g) | | | | | |
| Time until | 14.5 hours | 15 hours | 16.5 hours | 16.5 hours | 18.5 hours |
| Koji | | | | | |
| temperature | | | | | |
| reached to | | | | | |
| 35° C. | | | | | |

From the above result, in the case of using the preculture material cultured at 2×10⁶ spore/g for 16 hours (Comparative Example 4), it took 18.5 hours until the Koji was reached to 35° C. On the other hand, in the case of using the pre-cultured seed Koji precultured at 1×10⁷ spore/g for 18 hours or more (Examples 4 to 7), the Koji temperature was reached to 35° C., demonstrating that the Koji mold grew well.

### Example II

### Production of Soy Sauce in the Cases of Preculture and Conventional Method

Soy sauce was actually brewed using the pre-cultured seed Koji and its quality was examined.

(1) Preculture

The preculture conditions were the same as Example 1. That is, the pre-cultured seed Koji was obtained by culturing Aspergillus oryzae spores at 3×10⁶ spores/g on the steamed, water-soaked wheat bran prepared by adding 0.7 L water to 1 kg of wheat bran, at 28° C. for 24 hours.

### (2) Koji Production and Brewing

Water-soaked defatted soybeans prepared by adding 20 kg of water to 15 kg of defatted soybeans were steamed in a conventional manner. In addition, 14 kg of raw wheat was roasted and cracked according to a conventional method.

In Example 8, the steamed soybean, the crushed wheat and the pre-cultured seed Koji obtained in the preculture were mixed to prepare the raw material for the Koji production and then to perform the Koji production for 26 hours according to a conventional method. The amount of the pre-cultured seed Koji was 4% by weight per Koji raw material (total amount of the defatted soybean and cracked wheat) (Total amount of Koji mold spores is 5.9×10⁹ spores).

In Comparative Examples 5 and 6, water-soaked defatted soybeans prepared by adding 20 kg of water to 15 kg of defatted soybeans were steamed in a conventional manner. In addition, 15 kg of raw wheat was roasted and cracked according to a conventional method. Aspergillus oryzae spores instead of the pre-cultured seed Koji or the preculture material were inoculated into the raw material so as to be 1×10⁶ spores/g to perform the Koji production for 26 hours (Comparative Example 5) and for 48 hours (Comparative Example 6) according to a conventional method (total amount of Koji mold spores is 5.0×10¹⁰ spores).

In Example 8, moromi was prepared by adding 1 L of salt water with salt concentration of about 25% (w/v) to 700 g of Koji sampled at 26 hours after the start of the Koji production. After the preparation, lactic acid bacteria and yeast were added to the mixture of the Koji and salt water according to a conventional method. This moromi was fermented and aged for 4 months.

In Comparative Examples 5 and 6, two kinds of moromi were prepared by adding 1 L of salt water with salt concentration of about 25% (w/v) to 700 g of Koji sampled at 26 hours (Comparative Example 5) and 48 hours (Comparative Example 6) after the start of the Koji production, respectively. After the preparation, lactic acid bacteria and yeast were added according to a conventional method to obtain two kinds of moromi. Each moromi was fermented and aged for 4 months. The Koji produced in 48 hours in Comparative Example 6 is referred to as soy sauce Koji produced as a so-called conventional method.

### Example III

### Comparison in Koji Production Process and Soy Sauce Brewing Process

### (3-1) Enzyme Activity in Koji

In the Koji obtained in Example 8 and Comparative Examples 5 and 6, protease, glutaminase, leucine aminopeptidase and α-amylase activity which are known to affect ingredients and taste of soy sauce were measured. The enzyme activity in Koji was measured according to “Soy Sauce Test Method” (Japan Soy Sauce Research Institute, 1985). The results are shown in Table 3. Each enzyme activity is shown as a relative value with enzyme activity in Comparative Example 6 (48-hour Koji production) which is a conventional method, as 1.00.

TABLE 3

| | | | Leucine | |
|---|---|---|---|---|
| | Protease | Glutaminase | aminopeptidase | α-Amylase |
| | activity | activity | activity | activity |
| Comparative | 1.00 | 1.00 | 1.00 | 1.00 |
| Example 6 (48 hr) | | | | |
| Comparative | 0.44 | 0.44 | 0.55 | 0.81 |
| Example 5 (26 hr) | | | | |
| Example 8 (26 hr) | 1.25 | 0.82 | 1.51 | 1.02 |

As a result, in the case of 26-hour Koji production (Comparative Example 5) not using the pre-cultured seed Koji or the preculture material, sufficient enzymatic activity could not be obtained. In the case of using the pre-cultured seed Koji as Example 8, sufficient enzyme activities of protease, glutaminase, leucine aminopeptidase, and α-amylase were found in even 26-hour Koji production.

### (3-2) Ingredient Analysis of Soy Sauce

Subsequently, each moromi of Example 8 and Comparative Examples 5 and 6 after four months of the preparation (after fermentation/aging for 4 months) was squeezed and the concentration of each ingredient in the resulting soy sauce was measured. The concentration of each ingredient was measured according to the method described in Soy Sauce Test Method. The results are shown in Tables 4 and 5. All of the concentrations (%) in the table indicate w/v.

TABLE 4

| | % Total | | | % Reducing | % Glutamic |
|---|---|---|---|---|---|
| | nitrogen | % Salt | % Alcohol | sugar | acid nitrogen |
| Comparative | 2.12 | 17.5 | 2.06 | 3.52 | 0.14 |
| Example 6 | | | | | |
| (48 hr) | | | | | |
| Comparative | 1.90 | 16.6 | 1.93 | 4.69 | 0.09 |
| Examples 5 | | | | | |
| (26 hr) | | | | | |
| Example 8 | 2.03 | 17.0 | 2.26 | 3.40 | 0.13 |
| (26 hr) | | | | | |

TABLE 5

| | % Lactic acid | % Acetic acid |
|---|---|---|
| Comparative | 1.47 | 0.26 |
| Example 6 | | |
| (48 hr) | | |
| Comparative | 1.20 | 0.29 |
| Examples 5 | | |
| (26 hr) | | |
| Example 8 | 1.31 | 0.25 |
| (26 hr) | | |

In the soy sauce prepared by using the Koji of Comparative Example 5 (26-hour Koji production), the amount of glutamic acid nitrogen and the amount of total nitrogen remarkably decrease whereas the amount of reducing sugar increases, and there were remarkable differences in values of other ingredients, in comparison with the soy sauce prepared by using the Koji of Comparative Example 6 (48-hour Koji production).

On the other hand, the values of the ingredients corresponding to those of the soy sauce according to the conventional method using the Koji of Comparative Example 6 (48-hour Koji production) were shown from the soy sauce using the preculture method of Example 8 (i.e., Example 1), even when the soy sauce was produced by using the Koji of 26-hour Koji production. In this way, it was found that the values of the ingredients equivalent to those of the soy sauce according to the conventional method could be obtained by using the pre-cultured seed Koji of Example 8 even when the Koji production time was remarkably shortened to 26 hours.

### (3-3) Nitrogen Utilization Rate

Total nitrogen in soy sauce recovered after squeezing against total nitrogen in the moromi was measured to calculate nitrogen utilization rate. The results are shown in Table 6. Numerical values in the table indicate relative values with the nitrogen utilization rate in the case where the Koji of Comparative Example 6 (48-hour Koji production) was used, as 1.

TABLE 6

| | Nitrogen |
|---|---|
| | utilization rate |
| Comparative | 1.00 |
| Example 6 | |
| (48 hr) | |
| Comparative | 0.97 |
| Example 5 | |
| (26 hr) | |
| Example 8 | 1.00 |
| (26 hr) | |

In the case of the preparation of the soy sauce using the Koji of Comparative Example 5 (26-hour Koji production), the nitrogen utilization rate decreased by about 3% in comparison with that of 48-hour Koji production as the conventional manner (Comparative Example 6). On the other hand, it was found that the nitrogen utilization rate equivalent to that of the conventional 48 hour-Koji production (Comparative Example 6) could be obtained by using the pre-cultured seed Koji of Example 8 even when 26 hour-Koji production was performed.

### (3-4) Organoleptic Evaluation

The obtained soy sauce was adjusted to have total nitrogen content of 1.57% (w/v), sodium chloride of 16.6% (w/v) and alcohol of 2.2% (w/v) and was heated using boiling water for 10 minutes. The resulting heated soy sauce was poured in a measuring cylinder to settle on the lees at 50° C. for 2 days.

After centrifugation of the above-mentioned heated soy sauce to remove the lees, an organoleptic test was carried out. In the organoleptic test, it was tested whether soy sauce (control soy sauce) using the Koji of Comparative Example 6 (48-hour Koji production) could be distinguished with soy sauce using the Koji of Example 8 (26-hour Koji production) by a three-point discrimination method. The tests were conducted by 9 trained internal panelists.

As a result, it was found that there was neither significant difference nor distinguishability (2 out of 9) between the control soy sauce using the Koji prepared in Comparative Example 6 (48-hour Koji production) and the soy sauce using the Koji prepared in Example 8 (26-hour Koji production).

That is, it was found that a high-quality soy sauce having the organoleptic properties equivalent to the soy sauce prepared by the traditional 48-hour Koji production could be obtained by using the preculture method of Example 8 even when the Koji production time was remarkably shortened to 26 hours.

### (3-5) Conclusion

As described above, in the case of the Koji using the pre-cultured seed Koji of Example 8, Koji having the same enzymatic activity as the Koji obtained by performing the Koji production for 48 hours according to the conventional Koji production method could be obtained even when the Koji production time was remarkably shortened to 26 hours. Furthermore, it was revealed that soy sauce which has the equal ingredients and organoleptic properties as those of soy sauce obtained by the conventional method can be produced, by producing soy sauce with the Koji using the pre-cultured seed Koji of Example 8.

### Example IV

### Comparison in Koji Production Process

In the Koji production process using pre-cultured seed Koji, a more detailed study was conducted on the amount of the pre-cultured seed Koji to be added and the Koji production time.

### (1) Preculture

The pre-cultured seed Koji was obtained under the same preculture conditions as those in Example II.

Preculture conditions are the same as Example 1. That is, the pre-cultured seed Koji was obtained by culturing Aspergillus oryzae spores at 3×10⁶ spores/g on the steamed, water-soaked wheat bran prepared by adding 0.7 L water to 1 kg of wheat bran, at 28° C. for 24 hours.

### (2) Koji Production and Brewing

Water-soaked defatted soybeans prepared by adding 20 L of water to 15 kg of defatted soybeans were steamed in a conventional manner. In addition, 14 kg of raw wheat was roasted and cracked according to a conventional method.

In Examples 9, 10 and 11, the steamed soybean, the crushed wheat and the pre-cultured seed Koji obtained in the preculture were mixed to prepare a raw material for Koji production and then to perform the Koji production according to the conventional method. The amount of the pre-cultured seed Koji was 5% (Example 9), 7% (Example 10) or 10% (Example 11) by weight per Koji raw material (total amount of the steamed soybean and cracked wheat).

The Koji of Comparative Example 7 was produced by the same method as Comparative Example 5 in Example II.

### (3) Enzyme Activity in Koji

In the Koji obtained in Examples 9, 10 and 11 and Comparative Example 7, protease activity was measured at 14, 18, 22, 26, 30 and 48 hours after the start of the Koji production. Enzyme activity in Koji was measured according to “Soy Sauce Test Method” (Japan Soy Sauce Research Institute, 1985). The results are shown in Table 7. Each enzyme activity is shown as a relative value with enzyme activity in Comparative Example 6 (at 48 hours after the start of Koji production) which is a conventional method, as 1.00.

TABLE 7

| Preparation | | | | | | |
|---|---|---|---|---|---|---|
| amount of | Koji production time | | | | | |
| pre-cultured | 14 | 18 | 22 | 26 | 30 | 48 |
| seed Koji | hours | hours | hours | hours | hours | hours |
| 5% | 0.30 | 0.94 | 1.19 | 1.33 | 1.39 | 1.46 |
| (Example 9) | | | | | | |
| 7% | 0.28 | 0.94 | 1.11 | 1.25 | 1.42 | 1.58 |
| (Example 10) | | | | | | |
| 10% | 0.37 | 0.98 | 1.23 | 1.35 | 1.45 | 1.66 |
| (Example 11) | | | | | | |
| (Comparative | — | 0.09 | 0.27 | 0.63 | 0.75 | 1.00 |
| Example 7) | | | | | | |

As a result, in the case of Comparative Example 7 not using the pre-cultured seed Koji or the preculture material, protease activity was not sufficiently elevated unless 48 hours had elapsed from the start of the Koji production. On the other hand, in the Koji produced by the production methods of Examples 9, 10 and 11 in which the pre-cultured seed Koji was added in a weight ratio of 5, 7 and 10%, respectively, the protease activities were sufficiently improved at 18 hours from the start of the Koji production, and favorable Koji could be obtained. In addition, the high enzyme activities were maintained at even 48 hours after the start of the Koji production.

As described above, it was found that favorable Koji having sufficient protease activity could be obtained at 18 hours after the start of the Koji production, according to the production method of the present invention characterized by inoculating the pre-cultured seed Koji into Koji raw material.

1. A method for producing Koji for brewed foods, comprising the steps of:
(1) obtaining pre-cultured seed Koji by pre-culturing Koji mold using a culture material with 500 g/L or less of bulk specific gravity, and
(2) performing Koji production for 18 to 30 hours after inoculating the pre-cultured seed Koji into a protein raw material and/or a carbohydrate raw material.

2. The method according to claim 1, wherein the culture material for the pre-culture in the step (1) is derived from epidermal portions from grains or pseudo grains.

3. The method according to claim 1, wherein the culture material used for the pre-culture in the step (1) is derived from wheat brans.

4. The method according to claim 1, wherein in the step (1), the Koji mold is pre-cultured until the number of spores of the Koji mold is 1×10⁷ spores/g or more.

5. The method according to claim 1, wherein the brewed food is soy sauce or miso.

6. A method for producing a brewed food using the Koji produced by the method according to claim 1.
